(19) United States
(12) Patent Application Publication
(10) Pub. No.: US 2024/0009231 A1
(43) Pub. Date: Jan. 11, 2024

(54) Method for Pleurodesis and Delivery of Drugs to the Lung and Pleural Space

(71) Applicant: University of Cincinnati, Cincinnati, OH (US)

(72) Inventors: FRANCIS X MCCORMACK, Terrace Park, OH (US); YUSUKE TANAKA, Sapporo (JP)

(73) Assignee: University of Cincinnati, Cincinnati, OH (US)

(21) Appl. No.: 18/027,932

(22) PCT Filed: Sep. 27, 2021
(86) PCT No.: PCT/US21/52210

§ 371 Date: Mar. 23, 2023

Related U.S. Application Data
(60) Provisional application No. 63/083,376, filed on Sep. 25, 2020.
(60) Provisional application No. 63/151,105, filed on Feb. 19, 2021.
(60) Provisional application No. 63/194,246, filed on May 28, 2021.

(51) Int. Cl. A61K 33/42; A61K 9/00; A61K 9/16; A61K 47/69; A61P 11/00

(52) U.S. Cl. CPC A61K 33/42; A61K 9/007; A61K 9/1611; A61K 47/6923; A61K 9/0019; A61P 11/00

(57) ABSTRACT

A method to treat pneumothorax or malignant pleural effusion by pleurodesis in a mammalian subject is disclosed. The method involves administering particles to the lungs, the pleural space or both. The particles comprise a crystalline material that is non-toxic and is cleared over time by biodegradation. In some embodiments, the biodegraded crystalline material is readily cleared by the kidneys. In one embodiment, the crystalline material is essentially cleared from the lungs a few months after treatment.

### CROSS REFERENCE TO RELATED APPLICATIONS

This application is a continuation of PCT Application No. PCT/US21/52210 filed Sep. 27, 2021, which claims benefit of U.S. Provisional Application Ser. No. 63/083,376, filed Sep. 25, 2020, U.S. Provisional Application Ser. No. 63/151,105, filed Feb. 19, 2021, and U.S. Provisional Application Ser. No. 63/194,246, filed May 28, 2021, which applications are hereby incorporated by reference in their entirety.

### STATEMENT REGARDING FEDERALLY FUNDED RESEARCH OR DEVELOPMENT

This invention was made with government support under HL127455 awarded by the National Institutes of Health. The government has certain rights in the invention.

### TECHNICAL FIELD

The present invention relates to methods for pleurodesis.

### BACKGROUND OF THE INVENTION

Pleurodesis is a method used to treat pathological collection of air or liquid in the chest cavity, termed pneumothorax (collapsed lung) or pleural effusion (which can be malignant or benign), respectively, which pose daily management challenges in virtually every major hospital. Pleurodesis is accomplished by interventions that induce fusion of the pleura covering the lung (visceral pleura) and the chest wall (parietal pleura or submucosal tissues), and which obliterate the pleural space and prevent future accumulation of air or liquid. Pleurodesis can be achieved using surgical or chemical methods. Surgical methods include mechanical abrasion of the parietal pleural surface using gauze; pleurectomy in which strips of parietal pleura are removed to expose the fusogenic underlying tissues; and talc poudrage, in which talc is blown into the chest cavity as an aerosol to coat the visceral and parietal pleural surfaces under direct visualization in the operating room. These procedures can be performed through open thoracotomy or endoscopically via video-assisted thoracoscopy (VATS), and generally mechanical abrasion via VATS is the preferred surgical approach because it is less invasive and takes less time to recover from than pleurectomy or talc poudrage. Pleurodesis can also be performed by instilling fusogenic substances through a thoracostomy tube (chest tube), including liquid doxycycline (previously liquid tetracycline until it became unavailable), betadine (rarely used), bleomycin (rarely used) or talc slurry. In these non-surgical procedures, the patient has a chest tube placed between the ribs using local anesthesia and is serially positioned in prone, supine, and right and left lateral positions after instillation to gravitationally distribute the substance and enhance the likelihood of fusion of all surfaces. Doxycycline is the most widely used agent for chemical pleurodesis, but is also the least fusogenic, and is often associated with recurrences. Whether delivered surgically or through the chest tube, talc is considered the most fusogenic and most aggressive method of pleurodesis, and is often the approach of last resort for recurrent pneumothoraces or effusions. Talc is also problematic, because it is a magnesium silicate that is not degradable, can disseminate to other tissues including the liver and the eye, and persists in the body in inflammatory foci that can be associated with chronic pain and fatigue. In the event of the need for a future lung transplant, prior talc pleurodesis can predispose the patient to excess intraoperative bleeding because of the difficulty with identifying tissue planes after talc induced pleural fusion. In addition, contamination of talc with asbestos or direct oncogenic effects of talc have been the subject of litigation, and have resulted in >$5 B in settlements for patients with ovarian cancer who had used talc containing baby powder. For these reasons, clinical grade talc has become increasingly difficult to acquire for pleurodesis, and there is a desperate need to find acceptable alternatives to talc and doxycycline.

### SUMMARY OF THE INVENTION

To address this need, the present invention provides an improved method to treat pneumothorax or malignant pleural effusion by pleurodesis in a mammalian subject. The method involves administering particles to the lungs, the pleural space or both of the subject. The particles comprise a crystalline material that is non-toxic and is cleared over time by biodegradation. In some embodiments, the biodegraded crystalline material is readily cleared by the kidneys. In one embodiment, the crystalline material is essentially cleared from the lungs a few months after treatment. In another embodiment, the crystalline material is essentially cleared from the lungs by the end of three months after treatment. In another embodiment, the crystalline material is essentially cleared from the lungs by the end of one month after treatment. In one embodiment, the particles are selected from the group consisting of hydroxyapatite, β-Tricalcium phosphate, biphasic calcium phosphate, calcium sulfate, carbonate apatite, monocalcium phosphate monohydrate, dicalcium phosphate, dicalcium phosphate dihydrate, octocalcium phosphate, precipitated hydroxyapatite, monocalcium phosphate, α-tricalcium phosphate, sintered hydroxyapatite, oxyapatite, tetracalcium phosphate and combinations thereof In another embodiment, the particles are biodegradable hydroxyapatite (HA).

In one embodiment, the crystalline material is spherical. In another embodiment, the crystalline material is amorphous. In one embodiment, the crystalline material is sintered. In another embodiment, the crystalline material is bound or adsorbed to another fusogenic substance that promotes pleural symphysis. In one embodiment, the crystalline material is bound to another fusogenic substance selected from the group consisting of doxycycline, tetracycline, bleomycin, iodopovidine and combinations thereof.

In another embodiment, the HA is bound or adsorbed to another fusogenic substance that promotes pleural symphysis. In one embodiment, the HA is bound to another fusogenic substance selected from the group consisting of doxycycline, tetracycline, bleomycin, iodopovidine and combinations thereof. In another embodiment, the mammalian subject is human.

In another embodiment, the present invention is a method of delivering drugs to the lungs of a mammalian subject. The method involves linking or adsorbing one or more drugs to particles and administering the linked particles to the lungs, the pleural space or both of the subject. The particles comprise a crystalline material that is non-toxic and is cleared over time by biodegradation. In some embodiments, the biodegraded crystalline material is readily cleared by the kidneys. In one embodiment, the particles are selected from the group consisting of hydroxyapatite, β-Tricalcium phosphate, biphasic calcium phosphate, calcium sulfate, carbonate apatite, monocalcium phosphate monohydrate, dicalcium phosphate, dicalcium phosphate dihydrate, octocalcium phosphate, precipitated hydroxyapatite, monocalcium phosphate, α-tricalcium phosphate, sintered hydroxyapatite, oxyapatite, tetracalcium phosphate and combinations thereof In another embodiment, the particles are biodegradable hydroxyapatite (HA). In another embodiment, the HA is bound or adsorbed to another fusogenic substance that promotes pleural symphysis. In one embodiment, the HA is bound to another fusogenic substance selected from the group consisting of doxycycline, tetracycline, bleomycin, iodopovidine and combinations thereof

In one embodiment, the drugs are selected from the group consisting of antibiotics, chemotherapeutic agents and combinations thereof. In another embodiment, the crystalline material is bound or adsorbed to another fusogenic substance that promotes pleural symphysis. In one embodiment, the crystalline material is bound to another fusogenic substance selected from the group consisting of doxycycline, tetracycline, bleomycin, iodopovidine and combinations thereof. In another embodiment, the mammalian subject is human.

### BRIEF DESCRIPTION OF THE DRAWINGS

The foregoing summary, as well as the following detailed description of preferred embodiments of the application, will be better understood when read in conjunction with the appended drawings.

FIG. 1A is a graph showing the volume of pneumothorax air space in the thoracic cavity of mice treated with talc.

FIG. 1B is a graph showing the volume of pneumothorax air space in the thoracic cavity of mice treated with hydroxyapatite.

FIG. 2A is a graph showing the clearance of talc particles over time as assessed by micro-computed tomography (“microCT”).

FIG. 2B is a graph showing the clearance of hydroxyapatite particles over time as assessed by microCT.

FIG. 3A is a graph showing the macroscopic pleural adhesion score for talc over time.

FIG. 3B is a graph showing the macroscopic pleural adhesion score for hydroxyapatite over time.

FIG. 3C is a graph comparing the adhesion scores of hydroxyapatite versus talc.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

As used herein, the term “biodegradable” means that the material can be chemically degraded or degraded in the body to form non-toxic components.

As used herein, the term “fusogenic” means capable of mediating membrane fusion.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In one embodiment, the present invention is an improved method to treat pneumothorax or malignant pleural effusion by pleurodesis that involves the use of hydroxyapatite (HA) as the fusogenic agent applied to pleural surfaces. In another embodiment, the present invention uses HA particles as drug delivery agents. In one embodiment, the present invention comprises a method for pleurodesis and drug delivery using a non-toxic and biodegradable form of HA.

In another embodiment, we describe an improved method for pleurodesis that involves the use of crystalline materials that are non-toxic and are cleared over time by biodegradation that are readily cleared by the kidneys. In one embodiment, the crystalline materials are cleared over time by biodegradation into calcium and phosphate ions. In one embodiment, the crystalline material is essentially cleared from the lungs by the end of one month after treatment. In some embodiments, the crystalline material is sintered, in other embodiments it is not sintered. In some embodiments, the crystalline material is bound or adsorbed to at least one other fusogenic substance known to promote pleural symphysis. Examples of such fusogenic substances include doxycycline, tetracycline, iodopovidine iodine and bleomycin. In other embodiments, the crystalline material is not bound or adsorbed to another fusogenic substance.

In some embodiments, the crystalline material is amorphous. In other embodiments, the crystalline material is spherical. In some embodiments, the crystalline material is used in conjunction with additives to improve adherence to or distribution across the pleural surface.

In one embodiment, the crystalline material comprises particles that are selected from the group consisting of hydroxyapatite, β-Tricalcium phosphate, biphasic calcium phosphate, calcium sulfate, carbonate apatite, monocalcium phosphate monohydrate, dicalcium phosphate, dicalcium phosphate dihydrate, octocalcium phosphate, precipitated hydroxyapatite, monocalcium phosphate, α-tricalcium phosphate, sintered hydroxyapatite, oxyapatite, tetracalcium phosphate and combinations thereof

In another embodiment, we describe an improved method for delivering drugs to the lungs of a mammalian subject. The method involves linking or adsorbing one or more drugs to the crystalline material described above and administering the linked/adsorbed material to the lungs and/or pleural space of the subject. In one embodiment, the drugs are selected from the group consisting of antibiotics, chemotherapeutic agents and combinations thereof.

The present invention has found that when various particles, including HA and silicates including talc, asbestos and silica, are instilled into the lungs of mice via the trachea, they induce the recruitment of circulating monocytes and their subsequent differentiation into pulmonary osteoclast like cells (POLC). Without being bound by theory, we believe that the inflammation and fibrosis we observe in the lungs after instillation of these particles is due to collateral tissue damage induced by the secretion of acid and matrix degrading substances by POLC. There are differences in how the lung and pleural space respond to these particles depending on whether they are degradable. When nondegradable silica or asbestos is instilled into the lungs, monocytes are recruited that differentiate into osteoclasts, but the particles are not cleared, and persistent inflammation and progressive pulmonary fibrosis ensue. In contrast, when HA is intratracheally instilled, there is transient monocyte rich inflammation and osteoclast differentiation, but both the particles and the inflammation are cleared within a month, and there is no residual fibrosis. Without being bound by theory, it appears that the POLC are able to degrade HA and that persistent inflammation that leads to fibrosis fades as particles are cleared.

The combination of the osteoclastogenic and biodegradable properties of HA make it an ideal agent for pleurodesis. On the basis of the ability of POLC lung to clear HA particles, the same should occur in the pleural space after installation of HA. As shown in the Examples below, the instillation of HA microspheres into the pleural space of mice induces pleural adhesions and fusion, and the particles are progressively cleared over time. Because the mineral composition of HA is identical to bone, the breakdown products of the particles, calcium and phosphate, are non-toxic and readily cleared by the body. There is no risk of contamination by asbestos or other non-degradable silicates. HA spheres of various uniform sizes and approved for human use are commercially available. Also, they are relatively straightforward to manufacture at scale.

HA microspheres also have potential as drug delivery devices, since bisphosphonate moieties bind avidly to HA, and can be used link antibiotics, chemotherapeutic agents or other therapeutics to the HA particles. Bisphosphonate moieties that do not inhibit osteoclast function, and linkers that are pH or protease sensitive can be incorporated into this invention to allow for delivery without cellular toxicity to POLC or monocytes, and with tissue context specific drug release. In summary, one embodiment of the present invention is a biodegradable HA particle-based technique that can be used for pleurodesis or delivery of drugs into the lung or pleural space.

In another embodiment of the present invention, compositions of hydroxyapatite and drug modifications of hydroxyapatite are disclosed. In some embodiments, hydroxyapatite particles were prepared using micrometric aggregates of hydroxyapatite manufactured by the spray dryer (SD) technique by a number of vendors. With the SD technique, lower atomization pressure variation results in larger particle size. Conditions can be adjusted to produce spherical particles with an optimal mean diameter for pleurodesis in the range of 15 to 25 μm or greater, with narrow particle size distribution. Stoichiometric hydroxyapatite (Ca₁₀(PO₄)₆(OH)₂) is stable up to 1000° C. and is easily sterilized for human use. Levels of Pb contamination are typically less than 20 ppb.

Drug and biomolecules can be adsorbed or linked to the particle which serves as an inorganic drug carrier. For the purposes of pleurodesis, for instance, tetracycline or doxycycline can be adsorbed to the particle, since they both bind avidly to bone.

### EXAMPLES

### Example 1

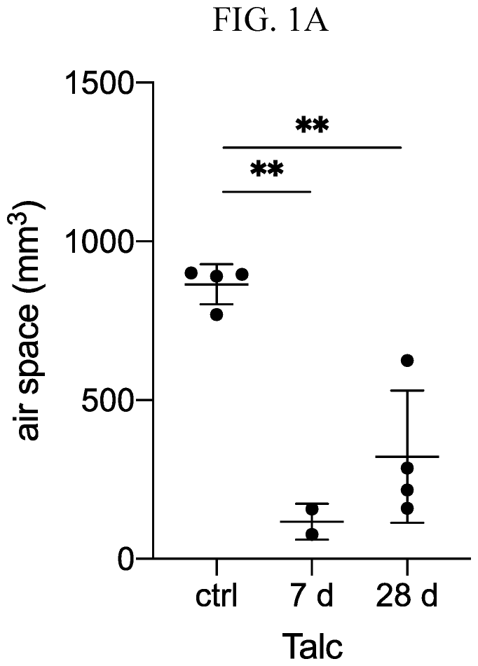

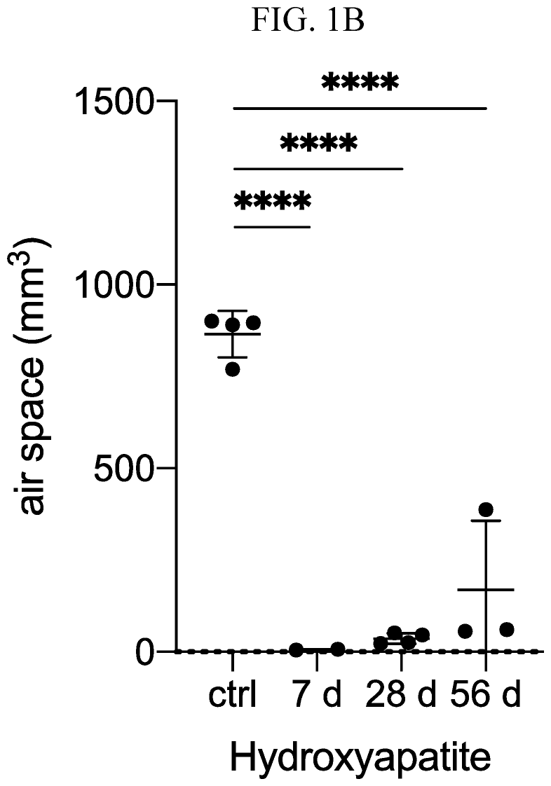

Either hydroxyapatite microspheres (HAMs, Fluidinova) or talc was injected via the intrapleural (i.pl.) route into C57BL/6J mice. For the mice injected with HAMs, 4 mg/g mouse of HAM/300 μL saline/mice was used. For the mice injected with talc, 2 mg/g mouse of talc/300 μL saline/mice was used. The mice were euthanized at day 0, day 7, day 14 or day 28 and post-mortem assessments were performed. Effectiveness of pleurodesis was assessed by introducing air into the pleural space. Each lung was punctured percutaneously with a 20 G needle for 12 times for left and right lung (total 24 times) (transthoracic needle puncture). Then, a CT scan was taken to measure the volume of pneumothorax air space in the thoracic cavity (see FIGS. 1A and 1B). In the figures, *p<0.05, **p<0.01, ***p<0.001, ****p<0.0001.

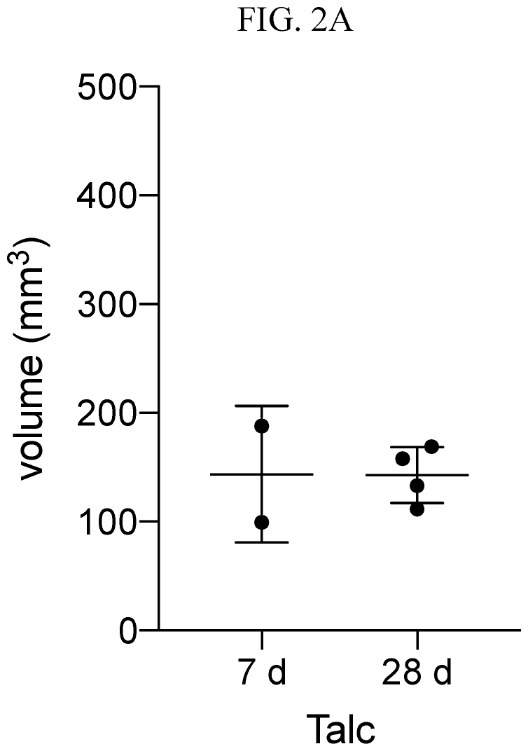

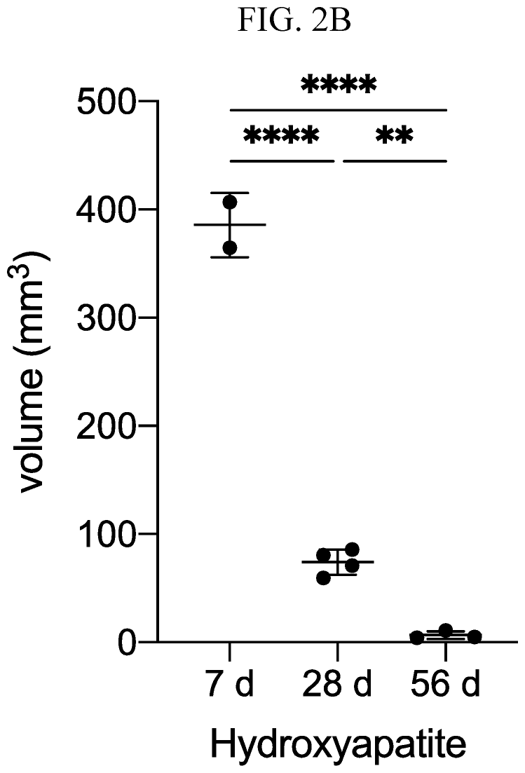

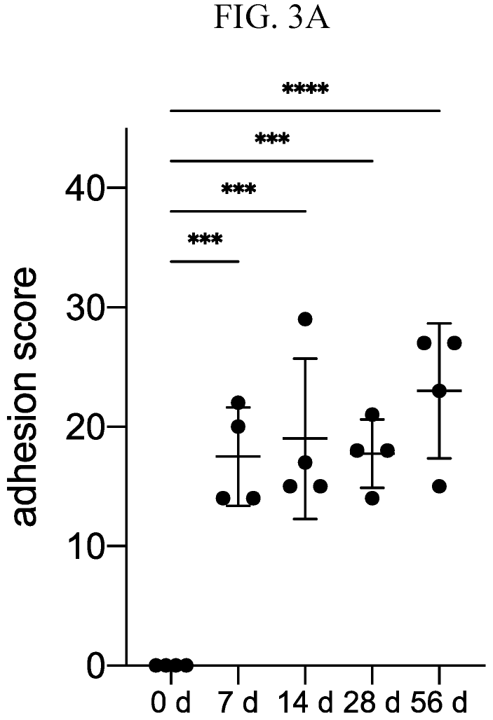

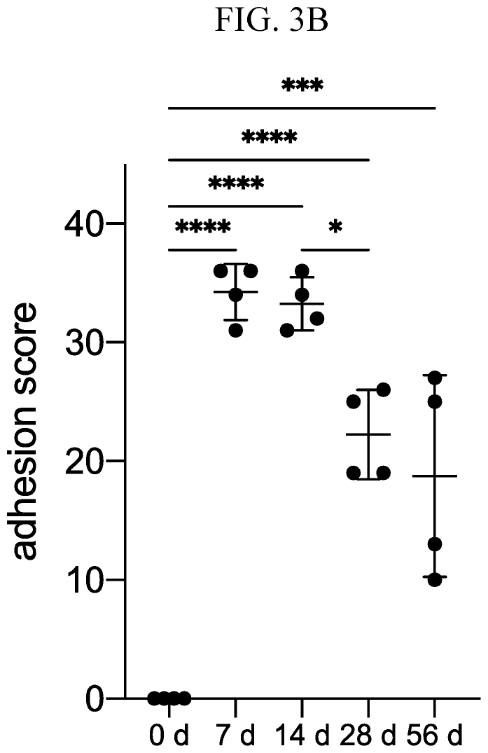

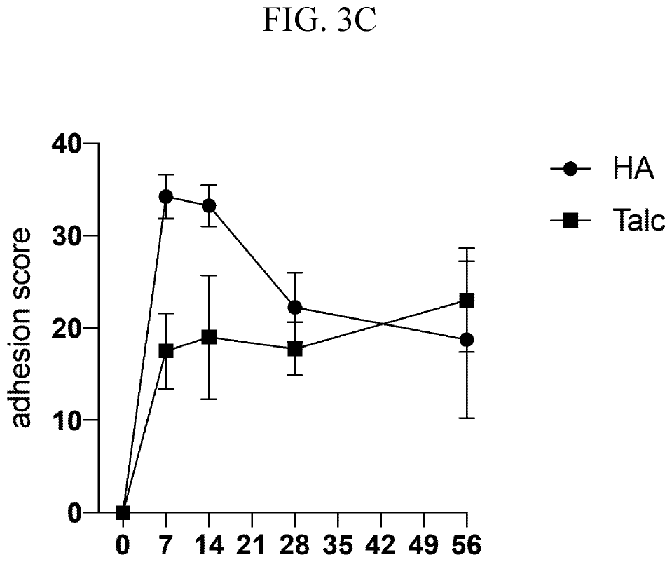

In addition, paraffin-embedded lung sections were stained with H&E and Mason's trichrome reagent for microscopic evaluation of pleural fibrosis and pleural thickness. Clearance of particles was assessed by microCT (see FIGS. 2A and 2B). Macroscopic pleural adhesions were scored (see FIGS. 3A, 3B and 3C); pleural lavage cells were collected to assess OLMNGC (osteoclast-like multinucleated giant cells) formation, including cytology, fibrosis- and osteoclast-related gene and protein expression of tartrate-resistant acid phosphatase (TRAP), (CTSK), and calcitonin receptor (Calcr) antibody by rtPCR and immunohistochemistry

The sclerosant actions of HAMs were comparable to talc in terms of pleural adhesion score, pleural thickness, and pneumothorax volume at day 28, and the pleural adhesion score was higher in the hydroxyapatite treated group on day 7 and 14. The amount of residual hydroxyapatite in the thoracic cavity decreased over time while talc did not. Osteoclastic transformation was observed in pleural lavage cells, as measured by increases in multinucleated TRAP, CTSK, and Calcr positive osteoclast-like cells and gene expression levels.

HAMs induce pleural adhesion that is comparable to talc, but unlike talc are almost completely cleared by day 28. HAMs are degradable, elementally pure particles capable of inducing an osteoclast response that show promise as superior sclerosants for pleurodesis.

### Example 2

Hydroxyapatite powder can be delivered using a pressurized canister. After aspiration of all pleural fluid, and lysis of adhesions to enhance access to all pleural surface, 2 to 10 grams of hydroxyapatite powder are insufflated into the pleural space. Hydroxyapatite powder can be administered with or without visualization. Visualization can be provided by video assisted thoracoscopy, or medical thoracoscopy, with appropriate modes of local or general anesthesia. Hydroxyapatite is delivered from pressurized canisters via a delivery tube inserted through a pleural trocar that has been placed into the pleural space. The aerosol is administered by pressing the button on the canister. The distal end of the delivery tube is pointed in several different directions while short bursts are administered, in order to evenly distribute the hydroxyapatite powder onto all accessible visceral and parietal pleural surfaces. Sudden decompression of the propellant gasses should be avoided due to sudden temperature drops that can cause pain in conscious patients. After hydroxyapatite insufflation, a chest tube is left in place, and negative pressure is applied (5 to 20 cm H₂O). The chest tube can be removed after 24 hours, or when pleural fluid drainage is less than 150 mL per day.

### Example 3

Alternatively, hydroxyapatite can be insufflated (usually under direct vision) by hand-driven bulb powder blower attached to a glass or plastic vial of hydroxyapatite particles. All other procedures are as outlined above for delivery by pressurized canister.

### Example 4

Hydroxyapatite can be delivered by slurry. The hydroxyapatite slurry is a nondissolving suspension of hydroxyapatite powder in saline. It is prepared by injecting a volume of 50 mL of sterile sodium chloride (0.9 percent) into the hydroxyapatite powder bottle using a 16-gauge needle attached to a 60 mL LuerLock syringe and swirling continuously. The contents of the bottle can then be aspirated back into a 60 mL syringe, or divided (25 mL each) in two 60 mL syringes, and additionally diluted with 25 mL of sodium chloride in each syringe. The slurry should be injected through an indwelling chest tube within 12 hours of preparation.

### Example 5

Sedation and analgesia for the procedure. Once full expansion of the lung has been documented with a radiograph and the hydroxyapatite slurry prepared, an intravenous analgesic (eg, morphine,) and an anxiolytic/amnestic (eg, midazolam) are administered. Administration of 25 mL (250 mg) of 1 percent lidocaine intrapleurally by aerosol a few minutes before hydroxyapatite slurry administration can reduce pain.

### Example 6

Installation of a hydroxyapatite slurry can be conducted as follows. The syringe(s) containing the hydroxyapatite slurry is continuously agitated to suspend the hydroxyapatite. After sterilizing the site of injection, the slurry (˜5-10 grams) is injected intrapleurally either via an injection into the chest tube proximal to a clamped section of tube or via a port of a three-way stopcock. The hydroxyapatite slurry initially distributes quite poorly over the pleural surfaces and tends to collect at the caudal sinuses. To distribute the hydroxyapatite slurry more evenly, supine, prone and right and left lateral positioning of the patient is recommended.

The chest tube should stay clamped for one hour; followed by negative 5 to 20 cm H₂O of active suction. The chest tube can be removed after 24 hours, or when pleural fluid drainage is less than 150 mL per day.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of generating pleurodesis in a mammalian subject, the method comprising administering particles to the lungs, the pleural space or both of the subject wherein the particles comprise a crystalline material that is non-toxic and is cleared over time by biodegradation.

2. The method of claim 1 wherein the particles are selected from the group consisting of hydroxyapatite, β-Tricalcium phosphate, biphasic calcium phosphate, calcium sulfate, carbonate apatite, monocalcium phosphate monohydrate, dicalcium phosphate, dicalcium phosphate dihydrate, octocalcium phosphate, precipitated hydroxyapatite, monocalcium phosphate, α-tricalcium phosphate, sintered hydroxyapatite, oxyapatite, tetracalcium phosphate and combinations thereof.

3. The method of claim 1 wherein the particles are biodegradable hydroxyapatite (HA).

4. The method of claim 1 wherein the crystalline material is spherical.

5. The method of claim 1 wherein the crystalline material is amorphous.

6. The method of claim 1 wherein the crystalline material is sintered.

7. The method of claim 1 wherein the crystalline material is bound or adsorbed to another fusogenic substance that promotes pleural symphysis.

8. The method of claim 7 wherein the crystalline material is bound to another fusogenic substance selected from the group consisting of doxycycline, tetracycline, bleomycin, iodopovidine and combinations thereof.

9. The method of claim 3 wherein the HA is bound or adsorbed to another fusogenic substance that promotes pleural symphysis.

10. The method of claim 9 wherein the HA is bound to another fusogenic substance selected from the group consisting of doxycycline, tetracycline, bleomycin, iodopovidine and combinations thereof.

11. The method of claim 1 wherein the mammalian subject is human.

12. A method of delivering drugs to the lungs of a mammalian subject, the method comprising linking or adsorbing one or more drugs to particles and administering the linked particles to the lungs, the pleural space or both of the subject, wherein the particles comprise a crystalline material that is non-toxic and is cleared over time by biodegradation.

13. The method of claim 12 wherein the particles are selected from the group consisting of hydroxyapatite, β-Tricalcium phosphate, biphasic calcium phosphate, calcium sulfate, carbonate apatite, monocalcium phosphate monohydrate, dicalcium phosphate, dicalcium phosphate dihydrate, octocalcium phosphate, precipitated hydroxyapatite, monocalcium phosphate, α-tricalcium phosphate, sintered hydroxyapatite, oxyapatite, tetracalcium phosphate and combinations thereof.

14. The method of claim 12 wherein the particles are biodegradable hydroxyapatite (HA). The method of claim 12, wherein the drugs are selected from the group consisting of antibiotics, chemotherapeutic agents and combinations thereof.

16. The method of claim 12 wherein the crystalline material is bound or adsorbed to another fusogenic substance that promotes pleural symphysis.

17. The method of claim 16 wherein the crystalline material is bound to another fusogenic substance selected from the group consisting of doxycycline, tetracycline, bleomycin, iodopovidine and combinations thereof.

18. The method of claim 14 wherein the HA is bound or adsorbed to another fusogenic substance that promotes pleural symphysis.

19. The method of claim 18 wherein the HA is bound to another fusogenic substance selected from the group consisting of doxycycline, tetracycline, bleomycin, iodopovidine and combinations thereof.

20. The method of claim 12 wherein the mammalian subject is human.
